(19) United States
(12) Patent Application Publication
(10) Pub. No.: US 2024/0108786 A1
(43) Pub. Date: Apr. 4, 2024

(54) TISSUE ENGINEERED BONE GRAFT USED IN INFERIOR TURBINATE RECONSTRUCTION

(71) Applicant: SHANGHAI RESTHETIC BIO CO., LTD, Shanghai (CN)

(72) Inventors: Guangdong ZHOU, Shanghai (CN); Yu LIU, Shanghai (CN); Runjie SHI, Shanghai (CN); Chenyan JIANG, Shanghai (CN)

(21) Appl. No.: 18/258,630

(22) PCT Filed: Dec. 22, 2021
(86) PCT No.: PCT/CN2021/140551

§ 371 Date: Jun. 21, 2023

(30) Foreign Application Priority Data
Dec. 22, 2020 (CN) 202011525634.0

(51) Int. Cl. A61L 27/36

(52) U.S. Cl. CPC A61L 27/3608; A61L 27/365; A61L 27/3695; A61L 2430/02

(57) ABSTRACT

Provided is a tissue engineered bone graft used in inferior turbinate reconstruction. In particular, provided is a tissue engineered bone graft comprising a bone marrow stromal cell (BMSC) grown on a demineralized bone matrix. The tissue engineered bone graft is used in inferior turbinate reconstruction. The tissue engineering bone graft of the present invention may effectively reconstruct the inferior turbinate, has no immunogenicity, and has high safety.

### TECHNICAL FIELD

The present invention belongs to the field of biomedical tissue engineering, in particular to a tissue engineered bone graft for reconstruction of inferior turbinate.

### BACKGROUND

Empty nose syndrome (ENS) is an iatrogenic complication caused by overexcision of a turbinate. It is manifested as secondary atrophy of the nasal mucosa and a series of concomitational symptoms, including dryness of the nasopharynx, inability to concentrate, fatigue, irritability, anxiety and depression, etc. About 20% of patients with inferior turbinectomy will develop ENS. Due to the poor treatment effect, patients and their families are hostile to surgeons and hospitals, and even have fierce conflicts, which brings certain unstable factors to the society.

In order to treat ENS, scholars have tried to use a variety of filling materials, such as silicone, autologous or allogeneic bone, cartilage, artificial dermis, hydroxyapatite or compounds thereof. Although the above methods can achieve a certain therapeutic effect, but there are problems such as rejection, allergy, histocompatibility and so on.

In summary, there is an urgent need to develop a tissue-engineered bone graft for inferior turbinate reconstruction in this field.

### SUMMARY OF THE INVENTION

The present invention provides a tissue-engineered bone graft for inferior turbinate reconstruction.

In the first aspect of the present invention, it provides a tissue-engineered bone graft, which comprises:
(a) a decalcified bone matrix carrier;
(b) human bone marrow stromal stem cells (BMSCs); wherein,

The decalcification degree of the decalcified bone matrix carrier is 95-85%; and/or the thickness of the decalcified bone matrix carrier is 3-8 mm.

In another preferred embodiment, the decalcification degree of the decalcified bone matrix carrier is 92%-86%.

In another preferred embodiment, the thickness of the decalcified bone matrix carrier is 4.5-5.5 mm.

In another preferred embodiment, the BMSCs are autologous cells.

In another preferred embodiment, the BMSCs are derived from cancellous bone.

In another preferred embodiment, the cancellous bone comprises: ilium, sternum, ribs.

In another preferred embodiment, the graft is a solid cell-material composite, and the concentration of BMSCs in the composite is 1×10⁷ cells/cm³-1×10⁸ cells/cm³, preferably 2×10⁷ cells/cm³-7×10⁷ cells/cm³. The content of BMSCs in the composite is 1×10⁷ cells/g-1×10⁸ cells/g, preferably 2×10⁷ cells/g-7×10⁷ cells/g.

In another preferred embodiment, the shape of the tissue engineered bone graft corresponds to the shape of the inferior turbinate defect site that the human body needs to be transplanted.

In the second aspect of the present invention, it provides a method for preparing the bone graft in the first aspect of the present invention, which comprises steps:
(1) providing autologous BMSC cells derived from the autologous bone marrow;
(2) culturing BMSC cells by external expansion using culture liquid containing basic fibroblast growth factor (bFGF);
(3) inoculating BMSC cells in a decalcified bone matrix carrier, then being subjected to in vitro chondrogenic induction culture to form tissue-engineered bone (BMSC-decalcified bone composite).

In another preferred embodiment, in step (2), the in vitro culture medium is a low-glucose medium.

In another preferred embodiment, in step (2), the BMSCs are expanded to passages 2-5.

In another preferred embodiment, in step (2), the concentration of bFGF in the in vitro culture medium is 0-10 ng/mL; preferably 2-5 ng/mL.

In another preferred embodiment, in step (2), the expanded BMSC cells are long spindle-shaped, with small cell size and strong proliferative activity.

In another preferred embodiment, in step (3), the inoculation concentration of the BMSCs is 1×10⁷ cells/g-1×10⁸ cell s/g; preferably 2×10⁷ cell s/g-7×10⁷ cell s/g; more preferably, 3.5×10⁷ cell s/g-5×10⁷ cell s/g.

In another preferred embodiment, in step (3), the in vitro chondrogenic induction culture lasts for 0.5-8 weeks; preferably 0.5-4 weeks.

In the third aspect of the present invention, it provides a use of the bone graft of the first aspect of the present invention for preparing a medicant for repairing inferior turbinate defects.

In another preferred embodiment, the medicant is a material containing living cells.

In another preferred example, the inferior turbinate defect is selected from the base of the inferior turbinate, the peripheral tissue of the inferior turbinate, and the lateral wall of the nasal cavity.

In another preferred embodiment, the tissue engineered bone graft is also used to increase the volume of the inferior turbinate, reduce the volume of the nasal cavity, and improve nasal ventilation.

In the fourth aspect of the present invention, it provides a method of repairing the inferior turbinate defect site, wherein the bone graft of the first aspect of the present invention is administered to the subject in need.

It should be understood that within the scope of the present invention, the various technical features of the present invention above and the various technical features specifically described hereinafter (as in the examples) may be combined with each other to constitute a new or preferred technical solution. Due to space limitations, it is not repeated here.

### DESCRIPTION OF THE DRAWINGS

FIG. 1 shows the effect of the presence or absence of fibroblast growth factor (bFGF) in the medium on the growth of BMSC cells; wherein, in panel A, “+bFGF” refers to culture with bFGF, and “-bFGF” refers to culture without bFGF; in the line chart in panel B, the vertical axis represents the OD (optical density) value of the CCK-8 assay result, and the horizontal axis represents the number of days (d) of the culture.

FIG. 2 shows a sample of decalcified bone.

FIG. 3 shows the tissue-engineered bone formed after 4 weeks of in vitro osteogenic induction of BMSC-decalcified bone composite.

FIG. 4 shows a schematic diagram of the formation of the cartilage-like graft after 8 weeks of in vitro culture of BMSC inoculated with polyhydroxyacetic acid/polylactic acid scaffold material, wherein panel A is a cartilage-like graft, panels B, D and F are the histological, safranine-O, and type II collagen immunohistochemical staining of the graft (scale bar is 1 mm), and panels C, E and G are respectively enlarged in the black frame content in B, D and F (scale bar is 100 μm).

FIG. 5 shows a schematic diagram of tissue engineered cartilage formed by BMSC inoculated with polyglycolic acid/polylactic acid scaffold material after implantation in subcutaneous environment for 12 weeks. Wherein, panel A is the tissue engineered bone formed by the chondroid graft of BMSC-polyglycolic acid/polylactic acid tissue under the skin of nude mice, and panel B is the histological staining of panel A (scale of 100 μm).

FIG. 6 shows tissue-engineered bone tissue formed by transplanting the BMSC-decalcified bone composite into the subcutaneous environment of nude mice.

FIG. 7 shows the histological staining of the BMSC-decalcified bone composite.

FIG. 8 shows the preoperative and postoperative MRI images of the lateral wall of the nasal cavity equivalent to the inferior turbinate in patients with empty nose syndrome after implantation of BMSC-decalcified bone composite.

### DETAILED DESCRIPTION

Through extensive and intensive studies, the present inventors for the first time developed a tissue engineered bone based on a specific material of decalcified bone matrix, which is constructed with BMSC cells. The tissue engineered bone of the invention is especially suitable for repairing inferior turbinate defect site. The decalcified bone matrix in the present invention has a specific hardness and thickness: the appropriate thickness is conducive to supporting the inferior turbinate site, which is convenient for BMSC cells loading; specific hardness facilitates trimming while providing the strength needed for inferior turbinate support. In addition, BMSCs loaded on the material are beneficial to nasal mucosa repair. Specifically, by optimizing the thickness and decalcification degree of the decalcified bone carrier material and the in vitro culture conditions of BMSCs, the present inventors constructed a special tissue-engineered bone graft material to facilitate the reconstruction of the inferior turbinate. On this basis, the present invention has been completed.

The present invention is based on minimally invasive extraction for obtaining a small number of BMSC cells, through in vitro culture, the cells are inoculated with high-density in a decalcified bone material with specific size and thickness, then by culturing to obtain a bioactive BMSC cell-decalcified bone material composite. Relying on the osteogenesis of autologous tissue cells and the degradation and absorption of biological materials, and finally, a new inferior turbinate bone is formed.

### Terms

As used herein, “the tissue-engineered bone graft for the reconstruction of the inferior turbinate”, “tissue-engineered cartilage/bone graft of the present invention”, “cartilage/bone graft of the present invention” can be used interchangeably, all refer to the tissue-engineered bone graft for inferior turbinate repair described in the first aspect of the present invention.

In general, a decalcified bone-gelatin composite scaffold, polyglycolic acid/polylactic acid (PGA/PLA), polycaprolactone (PCL), or polycaprolactone composite hydroxyapatite can be used as a scaffold material for tissue-engineered bone.

The term “induction” refers to the process of providing a special biochemical environment to transform a cell population such as stem cells with multidirectional differentiation into another cell population with different functional characteristics.

The term “inoculation” refers to the process of evenly distributing cells on a three-dimensional scaffold material.

The term “autologous transplantation” refers to the process of removing the desired biological living material, such as bone marrow stromal stem cells, from an individual and administering it to the same individual.

In a preferred embodiment of the present invention, the material selected for a tissue engineering carrier is a decalcified bone matrix.

Decalcified Bone Matrix Carrier

Decalcified bone matrix (DBM) is a bone graft material that is decalcified by allogeneic bone or heterogeneous bone to reduce immunogenicity. The degree of decalcification corresponds to different mechanical strength. It has good biological characteristics, bone inductance, bone conductivity and biodegradability, promotes new bone formation and bone tissue mineralization, and then accelerates bone healing. It can effectively repair bone injury alone or combined with autologous bone, other biological materials and growth factors, and is an ideal scaffold material for bone tissue engineering.

In another preferred embodiment, the length of the decalcified bone material is 10-40 mm, preferably 34.5-35.5 mm; and the width of which is 5-15 mm, preferably 9.5-10.5 mm.

In one example of the present invention, the length, width and height of the decalcified bone material are 35 mm, 10 mm, 5 mm, respectively.

It should be understood that the size of the tissue-engineered bone graft of the present invention may be customized according to the situation of different patients. For example, pruning can be done according to the site of the inferior turbinate defect in different patients, and the actual requirements have been met.

The decalcified bone material in the tissue-engineered bone graft of the present invention requires a certain thickness in order to provide support at the specific location of the inferior turbinate. If the thickness is too large, it is not conducive to inoculation of stem cells and the full penetration of cell suspension, and the cultured cell-material composite will appear hollow phenomenon, which will affect the repair effect after implantation. If the thickness is too small, it does not meet the mechanical strength requirements, resulting in the loss of cell suspension during inoculation, and thus unable to effectively load cells, reducing the inoculation efficiency.

The present inventors optimize the thickness of the decalcified bone material, and in one preferred embodiment, the thickness of the decalcified bone material of the present invention is 1-8 mm; more preferably, the thickness is 2-5 mm.

Decalcification Degree

The source of bone tissue that can be used as a tissue engineering carrier of the present invention is not particularly limited, and may be allogeneic bone tissue derived from humans, or xenogeneic bone tissue derived from animals (such as pigs, cattle, sheep, dogs, etc.). Preferably, it is xenomorphic bone tissue derived from pigs or cattle.

Under the same treatment conditions, the toughness of the material is lower when the degree of decalcification is small, which will lead to the material fragmentation when trimming the material, increase the difficulty of operation, and prolong the degradation time of the material in the body. However, when the degree of decalcification is too large, the strength of the material is insufficient to meet the strength required to repair the inferior turbinate, which will affect the prognosis of the patient.

In one example of the present invention, the decalcification degree of the decalcified bone matrix carrier of the decalcified bone material of the present invention is 95-85%, preferably 92%-86%. That is, the calcium content of the decalcified bone material in the present invention should be controlled at about 5-15%, preferably 8-14%.

Methods of decalcification The bone tissue was immersed in liquid nitrogen for 5 min, and then placed in 75% ethanol solution for degreasing. The tissue was subsequently added into 0.5 M HCL solution for decalcification, and the HCL solution was replaced every two hours for a total of 3 times. After washing with deionized water for 3 times, 0.05% pancreatic enzyme solution was added and digested in a constant temperature shaker at 37° C. for 2 h for decellularization. Finally, it was freeze-dried to make decalcified bone matrix, and stored in a drying oven for later use.

Determination of Decalcification Degree

Measurements were made using plasma emission spectroscopy. 0.5 g of lyophilized decalcified bone was taken, fully grinded to make decalcified bone matrix powder, and it was put into a 100 ml volumetric flask. 5 ml of concentrated HNO₃ was added, digested at microwaved 190° C. for 18 minutes, and the volume was set to 100 ml; 1.5 ml solution was taken and added into plasma emission spectrometer (ICP) for detection, and the value was read. The experiment was repeated three times and the average was taken.

Bone Marrow Stromal Stem Cells

Bone marrow stromal cell (BMSC) is a class of tissue stem cells with multi-directional differentiation potential, which can differentiate into bone, cartilage, fat, muscle, nerve, tendon and ligament and other tissue cells in an appropriate induction environment in vitro and in vivo. The cell population has sufficient sources, convenient material collection and strong proliferation ability.

In one example of the present invention, the BMSCs were directly amplified with osteogenic induction solution, and after the cells reached a certain number, they were inoculated in decalcified bone or other tissue engineered scaffold materials, and continued to be cultured with osteogenic induction solution for 1-3 weeks.

In another preferred embodiment, the BMSCs were amplified with bFGF-containing low-glucose medium. bFGF can significantly increase the proliferative activity of BMSCs, which is beneficial to maintain their stem cell characteristics, thereby saving the number of required BMSCs and improving osteogenic activity.

In another preferred embodiment, a high concentration of BMSCs were inoculated in decalcified bone material, depending on the degree of atrophy of the patient's inferior turbinate to determine the size of decalcified bone to form a tissue-engineered bone (BMSC-decalcified bone composite).

The BMSC concentration of the present invention for inoculation is typically 1×10⁷ cells/g-8×10⁷ cells/g, preferably 2×10⁷ cells/g-5×10⁷ cells/g. Generally, the concentration of seed cells was adjusted by culture medium, and then mixed with the tissue-engineered carrier of the present invention, wherein the proportion of culture medium and solid material is not particularly limited when mixing, but the maximum amount of culture medium that can be adsorbed by the carrier of the present invention shall prevail.

In the graft of the present invention, various other cells, growth factors, various transgenic components may also be added or compounded, thereby maintaining cell phenotype, promoting cell growth or matrix synthesis ability, etc., or promoting tissue growth, blood vessels and nerve growth, etc.

The formed bone graft can be directly implanted in the bone defect and other sites of the body to repair the bone tissue defect or fill the bone tissue.

hBMSC-Decalcified Bone Composite

The cell inoculation concentration of the hBMSC-decalcified bone composite in the present invention is typically about 1×10⁷ cells/g-8×10⁷ cells/g or higher. The materials are decalcified bone materials or other solid materials, solid and liquid composite materials. The cell concentration is adjusted by the culture medium, and then mixed with the decalcified bone material, and the ratio of culture medium to decalcified bone material is not particularly limited when mixing, but the maximum amount of decalcified bone material that can adsorb the culture medium shall prevail. When the scaffold material is in a special three-dimensional shape, such as inferior turbinate, it is calculated according to the size of the actual volume.

Preparation Method

The tissue-engineered cartilage graft of the present invention is easy to make.

In one specific example, the BMSCs were first expanded with bFGF-containing low-glucose medium, and after the cells reached a certain number, they were inoculated in decalcified bone or other tissue-engineered scaffold materials, and then were cultured in vitro chondrogenic induction solution disclosed in Example 6 of the patent (ZL 201110268830.9) for 1-8 weeks to form a cartilage-like graft.

The main advantages of the present invention include:
(1) The decalcified bone matrix material of the present invention can effectively reconstruct inferior turbinate.
(2) The stem cells required by the present invention are derived from autologous cells, have non-immunogenicity and high safety.
(3) The BMSC microenvironment is conducive to the mucosal repair of the nasal cavity.
(4) The present invention requires only a small amount of stem cells, and the sampling process is routine operation without damaging normal tissues.
(5) The size of the graft can be prepared according to the shape of the tissue defect to achieve accurate repair.
(6) In vitro culture method is simple and easy to learn, easy to promote, and easy to form industrial products.
(7) The tissue-engineered cartilage/bone graft of the present invention has good plasticity and certain mechanical strength, is easy to be processed into the required shape and has a supporting function, and meets the requirements of the specific position of the inferior turbinate.

The present invention is further illustrated below in conjunction with specific example. It should be understood that the examples are not intended to limit the scope of the invention. The experimental methods in the following examples which do not specify the specific conditions are usually in accordance with conventional conditions, such as conditions described in Sambrook et al., Molecular Cloning: Laboratory Manual (New York: Cold Spring Harbor Laboratory Press, 1989, or as instructed by the manufacturers, unless otherwise specified. Unless otherwise stated, percentages and parts are by weight.

Osteogenic Condition Medium

medium), with the addition of 10 nmol/L of β-sodium glycmedium), with the addition of 10 nmol/L of β-sodium glycerol phosphate, 0.1 μmol/L of dexamethasone, 50 μmol/L of L-α-ascorbic acid phosphate, 300 mg/L of L-glutamine, 10 nmol/L of 1.25 (OH)₂VD₃ and 10% fetal bovine serum (hyclone, USA), wherein the reagents not specified are from Sigma Corporation, USA.

In Vitro Chondrogenic Induction Medium

The in vitro chondrogenic induction medium as disclosed in Example 6 of patent (ZL 201110268830.9).

BMSC Expansion Medium (Low-Glucose Medium Containing bFGF):

The medium used to expand BMSCs contains low-glucose DMEM medium 10 g, L-glutamine 300 mg, vitamin C 50 mg, and sodium bicarbonate 3.7 g per liter of liquid. Preferably, 2-5 ng/mL of basic fibroblast growth factor (bFGF) is added.

Osteogenic Induction Medium.

High-glucose DMEM culture medium containing 10% FBS, (3-glycerol phosphate 10 mM, vitamin D3 10 nM, dexamethasone 0.1 μM.

### Example 1: Acquisition and Cultivation of BMSCs

3-5 mL of bone marrow was taken from the patient's anterior superior iliac spine by puncture and placed on Percoll isolation solution (with a density of 1.073 g/L) for gradient density centrifugation. The ratio of bone marrow to isolation solution was 1:2. The cells were centrifuged at 2550 r/min for 30 minutes, the cloud cell layer in the middle were extracted, and cells were washed once with phosphate buffer (PBS). After centrifugation at 1550 r/min, the supernatant was discarded to obtain nucleated cells, and the cells were inoculated into a culture dish at 2×10⁷ cells/cm². The cells were expanded in vitro, and osteogenic induction was performed with osteogenic condition medium.

After 48 hours of primary cell inoculation, the medium was changed. After the cells reached 80%-90% confluent, they were digested by 0.25% trypsin and sub-cultured with 2×10³ cells/cm². The cells were placed in a 37° C., 5% CO2 incubator and cultured to the third passage, then the cells were collected and counted.

### Example 2: Optimization of BMSC culture conditions

The BMSCs were expanded with low-glucose medium containing bFGF. The medium used to expand BMSCs contains low-glucose DMEM medium 10 g, L-glutamine 300 mg, vitamin C 50 mg, and sodium bicarbonate 3.7 g per liter of liquid. The medium was supplemented with Ong, 1 ng/mL, 2 ng/mL, 5 ng/mL, 10 ng/mL basic fibroblast growth factor (bFGF). BMSCs were cultured with the above medium.

The results show that in the medium containing bFGF, the best concentration range of bFGF is about 2-5 ng/mL.

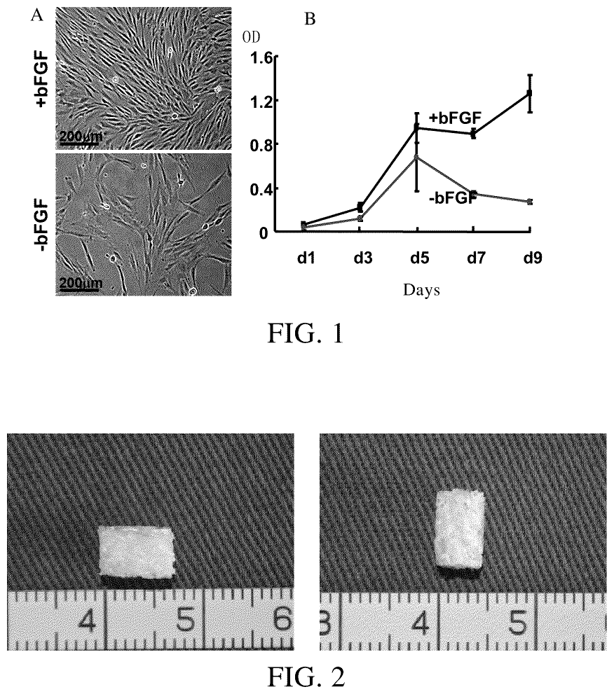

The morphological results of cells cultured with (+bFGF) and without (−bFGF) bFGF are shown in FIG. 1 panel A: the BMSC cultured with bFGF can maintain a longer spindle shape and have a smaller cell volume, while the morphology of BMSC expanded in the normal system without bFGF culture is more extensive.

As shown in FIG. 1 panel B, BMSCs cultured with bFGF can still have good proliferative activity on the ninth day of culture. The proliferation activity of BMSCs cultured without bFGF is continuously decreased after the fifth day of culture.

### Example 3: In vitro culture of BMSC composite tissue engineered scaffold material

(1) Experimental Methods:

Method 1, the BMSCs were directly expanded with osteogenic induction medium, and after the cells reached a certain number, they were inoculated in decalcified bone or other tissue engineered scaffold materials, and continued to be cultured with the above-mentioned osteogenic induction medium for 1-3 weeks.

Method 2: BMSCs were first expanded with low-glucose medium containing bFGF, and when the number of cells reached a certain level, they were inoculated in decalcified bone or other tissue engineered scaffold materials, and then cultured with osteogenic induction medium for 1-3 weeks.

Method 3: BMSCs were first expanded with low-glucose medium containing bFGF, and when the number of cells reached a certain level, they were inoculated in decalcified bone or other tissue engineered scaffold materials, and then cultured with in vitro chondrogenic induction medium preferred in Example 6 of patent (ZL 2011 1 0268830.9) for 0.5-8 weeks to form a chondroid graft (i.e., tissue engineered cartilage).

Among them, method 1 provides an osteogenic environment through osteogenic induction medium starting from the stage of cell expansion, which may be more conducive to subsequent bone regeneration.

In method 2, the cells were expanded in low-glucose medium containing bFGF, and more cells were obtained compared to method 1.

In method 3, based on method 2, chondrogenic induction medium was used to culture the cell-material composite for a longer period in vitro, and the regenerated tissue was more similar to cartilage and more tolerant to the surrounding environment after implantation into the inferior turbinate defect site, which is expected to improve the survival rate (The inferior turbinate defect site is in the submucosal microenvironment, and the blood supply is not very rich. It is not conducive to the long-term survival of general tissue engineered bone, but the tissue engineered cartilage is more tolerant to an ischemic environment). The above tissue-engineered cartilage implanted under the mucoperiosteal of the nasal cavity will further develop into bone tissue (matching the defect type) through “endochondral ossification” due to the terminal ossification of its internal BMSC.

Note: Method 3 is Better than Method 1 and Method 2.

(2) BMSCs were Inoculated in Decalcified Bone Material:

Decalcified bone was selected as the scaffold material for tissue engineered bone. Through decalcification treatment, the calcium content of decalcified bone material was controlled at about 8-10%. The size of decalcified bone material needs to be customized according to the situation of different patients. Generally, the length, width and height of a decalcified bone matrix carrier material are 35 mm, 10 mm and 5 mm, respectively, and the mass of the material is 4-5 g. The decalcified bone sample is shown in FIG. 2.

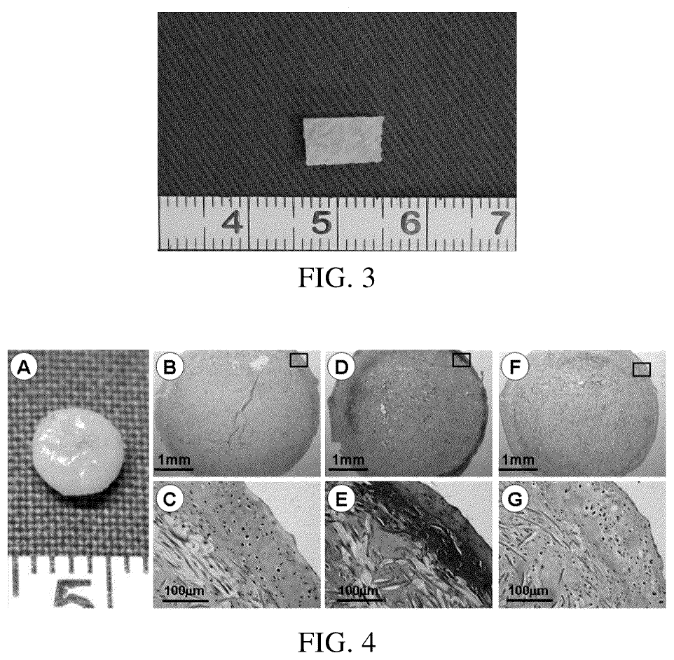

After osteogenesis induction, BMSCs were inoculated into decalcified bone material at a concentration of 3.5×10⁷ cells/g, and cultured for 0.5-8 weeks. The size of decalcified bone was determined by the degree of atrophy of the patient's inferior turbinate, and a BMSC-decalcified bone composite tissue engineered bone was formed. The BMSC-decalcified bone composite formed after 4 weeks of in vitro osteogenic induction is shown in FIG. 3.

(3) BMSCs were Inoculated on Other Materials:

BMSCs were expanded with low-glucose medium containing bFGF, and when the number of cells reached a certain level, they were inoculated into the poly-glycolic acid/polylactic acid scaffold material. After 0.5, 1, 2, 3, 4, 6, 8, or 12 weeks of culture with chondrogenic induction medium, chondroid grafts could be formed in vitro. The experimental result is shown in FIG. 4.

After 8 weeks of culture in vitro, the constructed product developed a porcelain white cartilaginous appearance (FIG. 4 panel A). Histological results showed a typical cartilage lacunar structure (FIG. 4 panel B, FIG. 4 panel C) and a rich expression of cartilage specific matrix, including glycosaminoglycan (FIG. 4 panel D, FIG. 4 panel E, red) and type II collagen (FIG. 4 panel F, FIG. 4 panel G, brown).

These results show that the constructed products obtained by culture with chondrogenic induction medium for 0.5-12 weeks have typical cartilage tissue characteristics.

### Example 4: Animal Transplantation Experiment

Cartilage-like grafts of BMSC-polyglycolic acid/polylactic acid scaffolds prepared in Example 3 (culture time 0.5, 1, 2, 3, 4, 6, 8, 12 weeks) were implanted under the skin of nude mice, respectively. A tissue-engineered bone sample developed after 12 weeks was shown in FIG. 5. The sample was taken and tested.

The result is shown in FIG. 5 panel A. The cartilage-like graft is tissue-engineered cartilage in vitro, and after implantation into the bodyBMSCs undergo terminal ossification, eventually forming tissue-engineered bone (hard bone). FIG. 5 panel B shows the histological staining of FIG. 5 panel A, which shows the typical bone-like tissue structure.

Among them: FIG. 5 panel A is a general view: tissue engineered cartilage regenerated in vitro can develop into bone-like tissue after implantation into a subcutaneously non-cartilage regeneration microenvironment;

FIG. 5 panel B shows HE staining: the histological staining result shows a typical bone-like structure.

The tissue-engineered bone formed after in vitro osteogenesis induction of BMSC-composite decalcified bone for 4 weeks (FIG. 3) was implanted subcutaneously in nude mice.

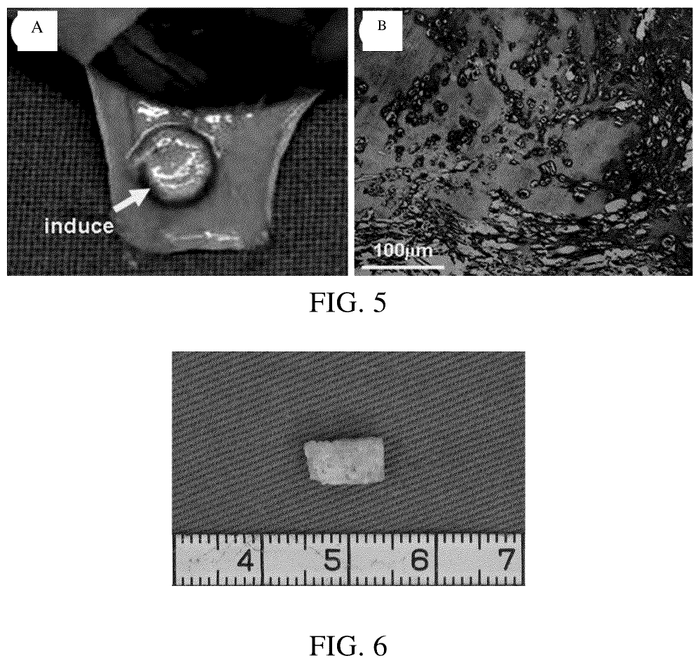

The result shows that tissue-engineered bone tissue is formed after 4-8 weeks of development. Among them, a sample of tissue-engineered bone tissue formed after 6 weeks of development is shown in FIG. 6.

Tissue-engineered cartilage regenerated in vitro was implanted into the subcutaneous environment (non-cartilage regeneration microenvironment), and the tissue engineered cartilage underwent ectopic ossification, forming tissue engineered bone. There was a non-cartilage regeneration microenvironment in inferior turbinate defect site, where the chondrogenesis-induced BMSC-decalcified bone complex can undergo ectopic ossification to form tissue-engineered bone.

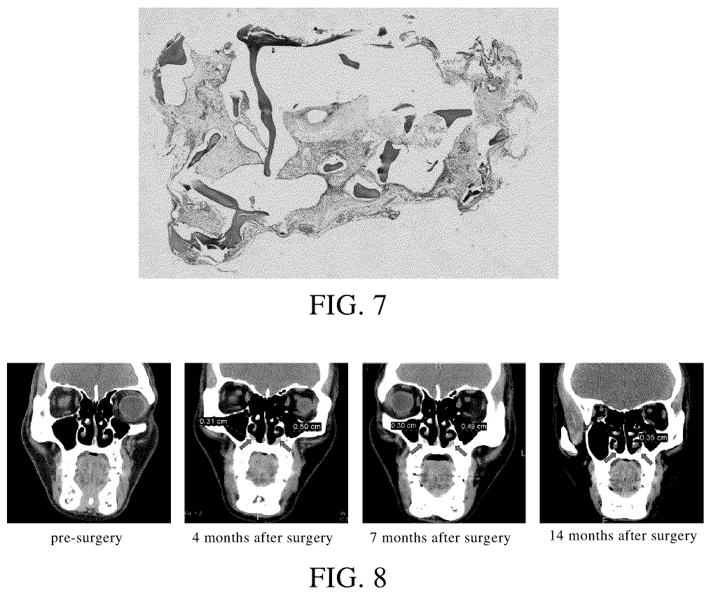

The histological staining of the BMSC-decalcified bone complex sample shown in FIG. 6 is shown in FIG. 7. The result shows that the BMSC-decalcified bone complex sample has a typical bone-like structure. Compared with FIG. 5 panel B, the tissue in FIG. 7 has a bone trabecular structure specific to bone tissue, which is more consistent with the tissue structure of natural bone tissue.

In addition, the terminal ossification of the constructed product cultured with chondrogenic-induction medium for 0.5-8 weeks is more sufficient, which can form hard bone with appropriate hardness. When the chondrogenic induction medium culture time 12 weeks, the constructed product still has cartilage tissue characteristics, but after implantation as a graft for the reconstruction of inferior turbinate, terminal ossification is not easy to occur through intrachondral ossification.

This suggests that the use of constructed product of 0.5 weeks to 4 weeks (or 3-30 days, or 7-28 days) is preferred, as on the one hand, it is cartilage-like during transplantation, making it easy to transplant; on the other hand, the graft is prone to terminal ossification through intrachondral ossification after transplantation, forming a tissue with shape and hardness more similar to that of the natural inferior turbinate.

(Note: If the time of in vitro chondrogenic induction exceeds 8 weeks, it will reduce the chance of ectopic ossification of the graft in vivo).

### Example 5: Inferior Turbinate Reconstruction

According to Method 1 in Example 3, cells were inoculated in decalcified bone material, and the tissue-engineered bone was formed by osteogenic induction. After In vitro culture for about 1-3 weeks, the cells and the biological material were well attached, the nasal mucosa was incised under nasal endoscopy, and the nasal mucosa and bone were separated to form an implantation cavity. The tissue engineered bone was pruned to a suitable shape outside the body and implanted into the volunteer's inferior turbinate or the outer wall of the nasal cavity, which is equivalent to the inferior turbinate.

Results: FIG. 8 shows that the volunteers' symptoms improved significantly, with significant improvements in imaging data and subjective scale scores.

The results indicate that since the graft is bone-like, it was convenient for transplantation. On the other hand, the graft of the present invention will eventually develop into mature bone tissue after transplantation, and the final formation shape and hardness are more similar to the natural inferior turbinate.

### DISCUSSION

The BMSC cells composite biomaterial used in the present invention has a number of advantages:
(1) Cells have multiple differentiation potentials and can differentiate into different tissues in specific differentiation environments. Therefore, it can directionally differentiate into bone tissue in the subnasal mucosal environment near the bone surface to achieve turbinate regeneration. Near the mucosa, it can directionally differentiate into mucosal tissue, and tissue repair can be carried out in a manner of cell replacement.
(2) Stem cells have a certain effect of paracrine, and can secrete VEGF to promote angiogenesis, IL-6 to regulate immune balance and inhibit inflammation, SDF to inhibit apoptosis in surrounding tissues, etc., which are conducive to repairing nasal mucosal function.
(3) Stem cells have a certain effect of immunomodulatory. They can prevent improper activation of T lymphocytes, inhibit the proliferation of T cells, and inhibit the differentiation of T cells to Th1 and Th17. In addition, T cells can be immunosuppressed by producing IDO catabolites. At the same time, stem cells can transform dendritic cells into tolerant phenotypes, and HLA-G5 and B7-H4 produced by stem cells can promote the differentiation of effector T cells into Treg (regulatory T cells), maintain T cell quiescence and regulate T cell subtypes balance. Stem cells can also inhibit the activation and function of B cells, and block these cells in the G0/G1 phase of the cell cycle through paracrine, thereby inhibiting the proliferation of B cells; they can also inhibit the differentiation of B cells into plasma cells by downregulating the mRNA expression of B lymphocytes induced mature protein-1 (Blimp-1). Therefore, stem cells can generate an immune tolerant microenvironment in the process of tissue repair, thereby interrupting the immune response, reducing the immune inflammatory response of the local microenvironment, and facilitating the repair of mucosal tissue.
(4) Compared with non-bioactive prosthesis implantation or cell-free loading implantation such as hydroxyapatite, stem cells have significant advantages in mucosal regeneration. Neither prosthesis implantation alone nor cell-free loading implantation can achieve the immunomodulatory effect of stem cell microenvironment, thereby reducing the regeneration efficiency of nasal septum mucosa after implantation, which affecting the prognosis of patients.

Compared with other materials, such as the use of autologous bone, or the use of cell-free graft hydroxyapatite for repair: autologous bone is autologous source and non-immunogenic, but its source is limited, which will cause secondary harm to patients, and some patients are difficult to accept. In addition, the content of viable cells in mature natural bone tissue after mineralization is very low, so it is difficult for the cells to survive when transplanted into the mucosal environment where the turbinate is located (lack of osteoblasts and blood supply). While the content of viable cells in tissue-engineered bone is much higher than that of natural bone, and the porosity is high when it is just implanted in the body, so nutrients can be obtained through body fluid penetration before vascularization is completed, and stable survival can be achieved after vascularization is established. Therefore, the survival rate of autologous tissue-engineered bone implantation (especially in non-osteogenic environments) is much higher than that of autologous bone implantation. Although cell-free grafts represented by bioceramics such as hydroxyapatite can improve turbinate state, ventilation and physiological structure, they have no obvious effect on nasal mucosal repair. In addition, due to the susceptibility of the nasal environment to infection and the lack of biological activity of artificial materials, there are many risks of material exposure, protrusion, infection and rejection. The degradation of BMSC-polyglycolic acid/polylactic acid bone graft after transplantation will produces acidic degradation products that will interfere with the regeneration of inferior turbinate.

Compared with the prior art, the BMSC-decalcified bone graft of the present invention has a certain degree of flexibility, which is convenient for trimming into a graft with suitable size and shape. Further, the graft of the present invention has excellent mechanical strength characteristics, and does not produce acid degradation products after transplantation, By providing a microenvironment conducive to bone regeneration, the graft of the present invention is easier to achieve bone regeneration and mucosal regeneration in the nasal mucosal environment, thus contributing to the formation of inferior turbinate with properties (such as hardness, etc.) that meet the requirements.

Therefore, the tissue-engineered bone graft of the present invention for inferior turbinate reconstruction has a significant repair effect, and has the characteristics of high safety, good compatibility, strong plasticity and excellent repair effect.

All literatures mentioned in the present application are incorporated by reference herein, as though individually incorporated by reference. In addition, it should be understood that after reading the above teaching content of the present invention, various changes or modifications may be made by those skilled in the art, and these equivalents also fall within the scope as defined by the appended claims of the present application.

1. A tissue-engineered bone graft, which comprises:
(a) a decalcified bone matrix carrier;
(b) human bone marrow stromal stem cells (BMSCs); wherein,
the decalcification degree of the decalcified bone matrix carrier is 95-85%; and/or
the thickness of the decalcified bone matrix carrier is 3-8 mm.

2. The bone graft of claim 1, wherein the decalcification degree of the decalcified bone matrix carrier is 92%-86%.

3. The bone graft of claim 1, wherein the thickness of the decalcified bone matrix carrier is 4.5-5.5 mm.

4. The bone graft of claim 1, wherein the BMSCs are autologous cells.

5. The bone graft of claim 1, wherein the BMSCs are derived from cancellous bone.

6. The bone graft of claim 5, wherein the cancellous bone comprises: ilium, sternum, ribs.

7. The bone graft of claim 1, wherein the graft is a solid cell material composite, and the concentration of BMSCs in the composite is 1×10⁷ cells/cm³-1×10⁸ cells/cm³, preferably 2×10⁷ cells/cm³-7×10⁷ cell s/cm³.

8. The bone graft of claim 1, the shape of the tissue engineered bone graft corresponds to the shape of the inferior turbinate defect site that the human body needs to be transplanted.

9. A method for preparing the bone graft of claim 1, which comprises steps:
(1) providing autologous BMSC cells derived from the autologous bone marrow;
(2) culturing BMSC cells by external expansion using culture liquid containing basic fibroblast growth factor (bFGF);
(3) inoculating BMSC cells in a decalcified bone matrix carrier, then being subjected to in vitro chondrogenic induction culture to form tissue-engineered bone (BMSC-decalcified bone composite).

10. The method of claim 9, in step (2), the concentration of bFGF in the in vitro culture medium is 0-10 ng/mL; preferably 2-5 ng/mL.

11. The method of claim 9, in step (3), the inoculation concentration of the BMSC is 1×10⁷ cells/g-1×10⁸ cells/g; preferably 2×10⁷ cells/g-7×10⁷ cells/g; more preferably, 3.5×10⁷ cells/g-5×10⁷ cells/g.

12. The method of claim 9, in step (3), the in vitro chondrogenic induction culture lasts for 0.5-8 weeks; preferably 0.5-4 weeks.

13. A use of bone graft of claim 1 for preparing a drug for preparing a medicant for repairing inferior turbinate defects.

14. The use of claim 13, the inferior turbinate defect is selected from the base of the inferior turbinate, the peripheral tissue of the inferior turbinate, and the lateral wall of the nasal cavity.

15. A method of repairing the inferior turbinate defect site, wherein the bone graft of claim 1 is administered to the subject in need.
